(19) United States
(12) Patent Application Publication
(10) Pub. No.: US 2007/0280949 A1
(43) Pub. Date: Dec. 6, 2007

(54) Combination Therapy for Gastroenteric Diseases Caused by Microorganisms

(76) Inventor: Michelle Alfa, Manitoba (CA)

Correspondence Address: Ade & Company, 1795 Henderson Highway, P.O.Box 28006, Winnipeg Menitoba, R2G OP1, CA

(21) Appl. No.: 10/564,057

(22) PCT Filed: Jul. 12, 2004
(86) PCT No.: PCT/CA04/01005

§ 371 Date: Jun. 26, 2007

Related U.S. Application Data
(60) Provisional application No. 60/485,722, filed on Jul. 10, 2003.

(51) Int. Cl. A61K 39/395; A61P 1/00

(52) U.S. Cl. 424/157.1

(57) ABSTRACT

The manufacture of a pharmaceutical composition comprising egg yolk antibodies and at least one probiotic and the use thereof to treat diseases caused by enteric pathogens is described.

### FIELD OF THE INVENTION

The present invention relates generally to the field of pharmaceutical compositions. More specifically, the present invention relates to a pharmaceutical composition for treating gastrointestinal disorders and the use thereof.

### BACKGROUND OF THE INVENTION

The widespread use of antibiotics began with the use of penicillin during World War II to treat infected wounds in soldiers. Discovered initially by Ernest Duchesne in 1896, and then rediscovered by Alexander Fleming in 1928, penicillin was hailed as a medical miracle, effectively neutralizing many types of disease-causing bacteria.

Today there are approximately 150 antibiotics approved for use in the US and Canada, that are prescribed for a wide spectrum of microbial infection and disease, including a number of gastroenteric diseases, including E. coli, Clostridium difficile, Salmonella, Campylobacter, and others.

While the use of antibiotics has proven to be a very effective means of treating these diseases over the past half century, this method has a serious drawback: over time, bacteria have the ability to develop resistance to virtually all forms of antibiotics. And when one strain develops a new resistance mechanism, the resistance is often quickly spread. The vast amount of antibiotics used by the livestock industry to maximize livestock growth and the over-prescription of antibiotics by physicians have both been implicated in increasing incidences of antibiotic resistance among a number of pathogenic microorganisms.

The effects of increased antimicrobial resistance are a decrease in the level of effectiveness of antibiotics in treating bacteria-induced diseases and an increase in the morbidity and mortality associated with bacterial infections

In response to the now recognized problem of antimicrobial resistance, the US Food and Drug Administration (FDA), along with a number of other governmental agencies, is now taking actions to address the problem, broadly including the following: responding to current threats from drug resistance, by taking actions to contain the spread of antibiotic resistant strains of bacteria; coordinating scientific research to address antimicrobial resistance; providing information on the appropriate use of antimicrobials to prolong the life of existing products; and facilitating and encouraging the development of products that help address the issue. This includes the development of alternatives to antibiotics, as well as the development of new antibiotics.

In addition to problems associated with general antibiotic resistance, the use of antibiotics has consequences for gastroenteric diseases, namely that the use of broad spectrum antibiotics has been implicated in the development of certain gastroenteric diseases—for example, Clostridium difficile associated diarrhea (CDAD).

CDAD is a side effect of using broad spectrum antibiotics because the ecological balance of the microflora in a patients gut is disturbed by the action of the antibiotic. Broad spectrum antibiotics indiscriminately kill both pathogenic strains of bacteria as well as beneficial strains of bacteria that would otherwise serve to suppress the pathogenic strains.

Overgrowth of C. difficile results from the organism being resistant to the antibiotic that is killing the beneficial gut organisms (as may occur after Clindamycin use), or from rapid germination of the C. difficile spore once the antibiotic is stopped (as may occur after therapy using Vancomycin). Spores are not affected by any antibiotic because they are not metabolically active.

One highly attractive and effective alternative to the use of antibiotics is the use of therapeutic antibodies to provide patients with passive immunity to pathogens that infect the gastrointestinal tract.

Antibodies are proteins formed in response to immunization, and are generally defined in terms of their specific binding to the immunizing antigen. Therapeutic antibodies are targeted antibodies that are administered to a patient to neutralize the effects of a specific pathogenic organism. Treatment with therapeutic antibodies usually involves the transfer of specific antibodies from an immunized individual to a non-immunized individual.

The advantages of using antibodies rather than antibiotics include the following:

Antibody treatments are sustainable. Antibodies in the immune system have been highly effective in providing protection for millions of years, and bacteria do not develop resistance to them. A mutation in a bacterium may result in different pathological properties but these can be readily compensated by the immune system through the production of complementary antibodies that are capable of neutralizing the new form of the microorganism.

Thus, use of antibodies to control diseases reduces the rate at which microorganisms become resistant to an antibiotic. Antibiotic resistance occurs as a result of natural selection. When an antibiotic acts on a population of microorganisms, some organism strains may survive. In the absence of competition from the other (susceptible) strains, the resistant strains become dominant, passing their resistant genes to subsequent generations. The use of antibodies in place of antibiotics avoids this process and thereby reduces the selective pressure toward antibiotic resistance.

The conventional method of commercial production of polyclonal antibodies for human therapeutics is in vivo production, in which large mammals (cattle, horses) are immunized against a pathogen, and antibodies are subsequently harvested from the immunized animal's blood. Disadvantages associated with this method of antibody production include the following: difficulty in purifying the antibodies of interest from other proteins in the serum; in vivo production is invasive, requiring that blood be drawn from the animal in order to harvest the antibodies produced; and fear of transmission of zoonotic diseases (e.g. Bovine spongiform encephalopathy, “mad cow disease”) through contact with serum.

An alternative means of producing polyclonal antibodies is in vivo production in birds (e.g., hens). In this method, chickens are immunized against a specific pathogen, and the antibodies are later harvested from eggs laid by the immunized hens. This method has been used for producing antibodies for use in the livestock feed industry. It is not in widespread use for the production of antibodies for human therapeutics, although there is significant scientific literature to support its use.

Egg yolk is recognized as a very good source of specific antibodies. The antibody, once produced in an immunized chicken can be obtained from the egg. The whole egg, its yolk or the purified immunoglobulin (IgY) can be used directly or processed (dried) and stored. The highest concentration of antibody is in the yolk.

Specific advantages offered over conventional antibody production include:

abundant supply—Hens produce large quantities of antibody relative to their body mass (more than 100 mg/egg), and hens typically lay 30-35 eggs per month.

ability to maintain high concentrations of specific antibodies in the laying hen over long periods—Hens can safely withstand higher doses of immunogen relative to their body weight than can large mammals.

low production cost—Overhead related to housing poultry is generally lower than for large mammals. Much of the harvesting of the antibodies (processing of the eggs) can be automated for economies of scale and reduced production costs.

less-invasive method of harvest—Antibodies may be obtained from eggs without harm to the chickens.

resulting antibodies can be easily stored—Egg yolk antibodies can be freeze-dried, stored and reconstituted using means well known in the art.

Avian therapeutic antibodies are currently used successfully in the livestock industry as feed additives to provide passive immunity to E. coli and other pathogens in commercial livestock.

As noted earlier, antibiotic use can predispose a patient to a gastroenteric disease by disrupting the normal balance of flora that typically colonize the gut. In response to this, an emerging alternative therapy is the use of probiotics to restore balance to the enteric ecosystem. A probiotic is an organism that contributes to the health and balance of the intestinal tract; also referred to as the “friendly”, “beneficial”, or “good” bacteria which when ingested acts to maintain a healthy intestinal tract and help fight illness and disease. Probiotic organisms can provide a protective effect only when a proper balance is maintained among all the different bacteria that normally reside in the intestine. Probiotic therapy is the recolonization of the gut with beneficial microorganisms to offset the overgrowth of pathogens and restore balance to the system.

The most frequently used probiotics are Lactobacilli and Bifidobacteria. The potential mechanisms of their action include competitive bacterial interactions, production of antimicrobial metabolites, mucosal conditioning, and immune modulation. However, probiotic treatments are plagued by poor reproducibility due to a lack of standardized studies. That is, the effectiveness of certain strains and/or concentrations at present appears to vary considerably.

### SUMMARY OF THE INVENTION

According to a first aspect of the invention, there is provided a pharmaceutical composition comprising:

polyclonal antibodies directed against at least one enteric pathogen; and

a probiotic.

According to a second aspect of the invention, there is provided a method of preparing a pharmaceutical composition comprising:

admixing a polyclonal antibodies directed against at least one enteric pathogen; and a probiotic.

According to a third aspect of the invention, there is provided a method of treating a gastrointestinal illness comprising administering to a patient in need thereof an effective dose of a pharmaceutical composition comprising:

polyclonal antibodies directed against at least one enteric pathogen; and

a probiotic.

### BRIEF DESCRIPTION OF THE FIGURES AND TABLES

FIG. 1 is a graph showing neutralization ability of IgY prepared by immunization of chickens with semi-purified Toxin A.

FIG. 2 is a graph showing ability of Lactobacillus GG to act as a probiotic and inhibit the growth of Clostridium difficile in the co-culture reactor vessel model.

FIG. 3 is a graph of Escherichia and Clostridium difficile in the co-culture reactor vessel model.

Table 1 is a summary of the IgY antibodies produced in avian host.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “enteric pathogen” refers to an organism capable of causing an infection in the gastrointestinal tract of an animal. Examples of enteric pathogenic microogranisms include but are by no means limited to Aeromonas hydrophilia, Bacillus cereus, Vibrio parahemolyticus, Vibrio cholerae O1, Vibrio cholerae non-O1, Vibrio vulnificus, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinla enterocolitica, Yersinia pestis, Yersinia pseudotuberculosis, Plesiomonas shigelloides, Listeria monocytogenes, enteric viruses, for example, rotavirus, Norwalk-like viruses, enteric adenoviruses, coronavirus and all other non-enveloped enteroviruses, and enteric parasites and fungi, for example, Cryptosporidium, and Cyclospora. It also refers to other microorganisms that may have no effect on the host but may have other undesirable effects, for example, methanogens or methane-producing organisms.

As used herein, “effective amount” refers to a dosage sufficient to have the desired effect. In some of the embodiments described below, “effective amount” refers to a dosage sufficient to treat or prevent a gastrointestinal illness, as described below. As will be appreciated by one of skill in the art, the specific dosage will vary according to, for example, the size and weight of the patient, the general condition of the patient, the severity of the illness, the enteric pathogen, the delivery method, and the titer of the antibodies.

As used herein, “patient” or “individual” refers to an animal, for example, a vertebrate. This includes but is by no means limited to a livestock animal, for example, poultry, cattle, swine and the like, house pets, and humans.

As described herein, the invention comprises a pharmaceutical composition comprised of a combination of polyclonal antibodies directed against at least one enteric pathogen and at least one probiotic microorganism. In some embodiments, the pharmaceutical composition is microencapsulated, as discussed below. In some embodiments, the pharmaceutical composition is in the form of a pill, tablet, capsule or other form for oral administration. In other embodiments, the pharmaceutical composition is in the form of a suppository, as described below. In yet other embodiments, the pharmaceutical composition is in combination with a suitable food product, for example, a yogurt or yogurt-based drink.

The probiotic is a beneficial microorganism, for example, a microorganism that is part of the microflora of healthy individuals. The probiotic may be selected from the group consisting of: Lactobacilli species, Bifidobacteria species, Saccharomyces species, Enterococci species, Eubacteria species and combinations thereof. As will be appreciated by one of skill in the art, other suitable probiotics known in the art may also be used within the invention.

As discussed above, in a preferred embodiment, the polyclonal antibodies are egg yolk antibodies derived from eggs from poultry immunized with at least one antigen derived from the targeted enteric pathogen, as discussed below. In a yet further preferred embodiment, the polyclonal antibodies comprise a powder made from the eggs or egg yolks from immunized poultry, for example, hens, or the polyclonal antibodies are isolated therefrom using means known in the art.

In use, as described below, the probiotic microorganisms or combination thereof establish within the GI tract of the animal, for example, a human patient, in need of treatment. However, the selected probiotic microorganisms are unable to colonize the GI tract. As a result, the probiotic effectively out-competes the enteric pathogen for nutrients and then is gradually replaced by gut flora microorganisms. It is further of note that the combination has a synergistic effect in that the egg material supports growth of the probiotic microorganism.

The anti-enterotoxigenic microorganism antibodies are preferably polyclonal antibodies. As discussed below, in some embodiments, the polyclonal antibodies are IgY antibodies derived from the eggs of immunized hens wherein the hens are immunized with at least one enteric pathogen antigen, as described below. As will be appreciated by one of skill in the art, as used herein, the term “antigen” is used broadly and refers to for example, but by no means limited to whole organisms, purified peptides, recombinant peptides, immunogenic peptide fragments and DNA vaccines encoding said antigen. As will be appreciated by one of skill in the art, the distinct advantage of polyclonal antibodies compared to monoclonal antibodies is that the polyclonal antibodies will include directed against a plurality of epitopes of the antigen.

As discussed below, in some embodiments, the polyclonal antibodies in the pharmaceutical composition may be antibodies raised against multiple targets from a single organism. As such, the antigenic targets may be, for example, but by no means limited to, whole bacteria, fimbriae, pilli, capsules, glycocalyces, secreted enzymes, for example, collagenase, hyaluronidase, coagulase and immunoglobin A protease, proteins isolated from the cell membrane, the lipopolysaccharide fraction, attenuated virus particles, viral proteins, cell surface proteins, for example, proteins involved in cellular adhesion or receptor binding, toxins and spores or immunogenic fragments thereof.

In yet other embodiments, the pharmaceutical composition may include polyclonal antibodies raised against more than one enteric pathogen.

For example, in these embodiments, for a pharmaceutical composition directed against C. difficile, the pharmaceutical composition may comprise antibodies purified from hens immunized against two or more of Toxin A, Toxin B, spore preps and C. difficile outer membrane proteins. In another example of these embodiments, wherein the pharmaceutical composition is arranged for treating E. coli O157:H7, the antibodies may be directed against two or more of verotoxins, flagellar proteins, and outer membrane proteins. As will be appreciated by one of skill in the art, in some embodiments, the polyclonal antibodies are directed against the organism itself and also against circulating and/or local toxins produced by the infecting organism, thereby increasing the efficacy of the treatment.

Thus, the pharmaceutical composition may include combinations of polyclonal antibodies raised against different antigens, for example, antibodies arranged to neutralize toxins, inhibit replication, and prevent spore outgrowth and probiotics arranged to outgrow in the gut, thereby overwhelming the enteric pathogen with other non-pathogenic organisms. It is of note that a considerable advantage of the instant pharmaceutical composition is that it is compatible with a wide range of antibiotics, meaning that there is no need to discontinue needed antibiotic therapy for a patient's underlying illness.

Thus, the above-described pharmaceutical composition prevents growth of the targeted enterotoxigenic microorganism (and at a number of life cycle stages, where appropriate) and promotes colonization of the gut by beneficial bacteria, as described below. This neutralizes and/or prevents bowel damage and inflammation, for example, PMC (pseudomembranous colitis) and also prevents recurrence or relapse.

As discussed above, the antibodies may be directed against for example pili, toxins, surface antigens, spores, viral particles or other antigenic targets thought to prevent replication.

For use, as discussed above, the pharmaceutical composition comprising a mixture of at least one probiotic microorganism and at least one polyclonal antibody preparation. The probiotic microorganism may be at a concentration of approximately 10⁸ to 10¹² colony-forming units. As will be appreciated by one of skill in the art, the titer of antibodies obtained from the eggs from the immunized hens may vary and will depend on for example the immune reaction of the hens to the antigen and environmental conditions. Thus, as will be apparent to one of skill in the art, it is important that the concentration or quantity of the polyclonal antibody preparation in the composition be an effective amount, the exact concentration or titer of which may vary as discussed above.

In some embodiments, the pharmaceutical composition comprises a combination of lyophilized probiotic microorganisms and egg powder. As will be apparent to one of skill in the art, methods of lyophilizing microorganisms are well-known in the art and in fact freeze-dried powders of the probiotic microorganisms described above are commercially available. Similarly, methods for preparing antibody-containing powder from eggs from immunized hens are well-known in the art.

In some embodiments, α-lactoalbumin or other oligosaccharides may be added to the pharmaceutical composition.

As discussed above, the pharmaceutical composition may be arranged to be administered to an animal, for example, a human patient in need of such treatment, in a variety of forms. As will be apparent to one of skill in the art, in this context, “in need of such treatment” refers to individuals infected with or believed to be infected with or at risk of developing an infection from an enteric pathogen. That is, the pharmaceutical composition may be used as a therapeutic or as a prophylactic, as described below.

For example, in some embodiments, the pharmaceutical composition is arranged to be administered orally, for example, in the form of a pill, tablet or capsule. As will be appreciated by one of skill in the art, in these embodiments, the pill, tablet or capsule may include for example binders and/or starches as well as other suitable additives known in the pharmaceutical arts. It is further of note that any suitable diluents, excipients or other such agents known in the pharmaceutical arts may be added as necessary.

As discussed herein, in these embodiments, the pharmaceutical composition may be encapsulated so that the composition is protected from gastric acidity. Specifically, in these embodiments, the coating is selected such that the pharmaceutical composition is arranged to be released under specific conditions, for example, released after a fixed period of time, that is, after sufficient time for the medicament to travel through the stomach and into the gastrointestinal tract or released on entry into the less-acidic GI tract from the stomach. It is of note that other suitable coatings may also be used provided that the medicament begins to dissolve on entry into GI tract and releases the pharmaceutical composition along the length of the GI tract.

One method of encapsulating pharmaceuticals is microencapsulation, which is a well understood process. Examples include U.S. Pat. Nos. 5,626,863; 5,601,760; 5,858,746; 6,528,093; 5,993,805; 6,309,569; 5,811,128; and 5,993,374, all of which are incorporated herein by reference.

In other embodiments, the pharmaceutical composition is arranged to be administered as a suppository. It is of note that methods of preparing a suppository are well understood in the art and that the above-described pharmaceutical combination can be readily adapted to be administered as a suppository using means known in the art.

In yet other embodiments, the pharmaceutical composition is incorporated into a food product, for example, a yogurt or yogurt-based drink. In these embodiments, the pharmaceutical composition would be administered as a nutraceutical. It is of note that methods of preparing such products are well known in the art and are well within the scope of the invention.

Ranging from mild annoyances to devastating dehydrating illnesses, acute gastrointestinal illnesses rank second only to acute upper respiratory illnesses as the most common diseases world-wide. Examples of gastrointestinal diseases include but are by no means limited to CDAD, food poisoning, gastroenteritis and antibiotic-associated pseudomembranous colitis. Wherein the pathogen is bacterial or fungal, the microorganism typically adheres to the gastrointestinal mucosa and then begins to compete with the bowel flora and colonize the mucosa. Once established, the enteric pathogens may activate host enzymes, such as adenylate cyclase or guanylate cyclase and/or may destroy intestinal mucosal cells. This in turn results in diarrhea and/or vomiting among other symptoms.

When administered to a patient, for example, an animal or individual in need of such treatment, that is, an individual or animal suffering from a gastrointestinal illness or at risk of developing a gastrointestinal disease as discussed herein, an effective dose of the pharmaceutical composition comprising polyclonal antibodies directed against at least one of the suspected pathogens and at least one probiotic will accomplish at least one of the following by inhibiting growth of the enteric pathogen and establishing competing probiotic organisms in the gastrointestinal tract: reduction of frequency of diarrhea and/or vomiting; greater symptom-free periods; and reduction of levels of enteric pathogen within the gastrointestinal tract.

It is of note that as discussed herein, the pharmaceutical composition may in some embodiments be coadministered with a suitable antibiotic. In these embodiments, the probiotic will be selected so that the probiotic is substantially immune to the antibiotic.

The platform encompasses the use of therapeutic polyclonal antibodies and probiotics as complementary components of the treatment. Additionally, the combined therapeutic agents (antibiotics and probiotics) are delivered using micro-encapsulation to ensure efficient delivery to the large intestine which is the site of the disease. The combination and efficient delivery of the two therapeutics as a single platform provides a robust and efficacious therapy that offers a realistic alternative to antibiotic therapy for CDAD and other gastroenteric diseases.

Because the therapy will incorporate components that will be derived from food products (egg yolks, naturally occurring yeast and bacterial cultures), the end product could be positioned as either a nutraceutical or a pharmaceutical.

In one exemplary embodiment, the technology is a multi-faceted therapeutic treatment to prevent and/or interrupt diarrheal disease due to C. difficile. The treatment platform will be comprised of the following:

(a) Therapeutic antibodies that target all critical stages in the disease process. Specific therapeutic actions will be: prevention of replication by the C. difficile organism; neutralization of the two toxins produced by the C. difficile organism (Toxin A and Toxin B); and prevention (or minimization) of spore replication.

(b) Nutraceuticals that restore normal bowel ecosystem balance and prevent the overgrowth of C. difficile. This includes the use of selected beneficial organisms that can replicate in the bowel and restore a normal balanced ecosystem which will prevent C. difficile overgrowth, as well as other components that will provide nutrition for the probiotic organisms and which may facilitate the production of end products (e.g. lactic acid) that control C. difficile.

In some embodiments, micro-encapsulation of the above components will ensure their delivery to the disease site without degradation due to exposure to the digestive tract. In these embodiments, this will increase efficacy of the treatment, using the most efficient dosing.

Thus, the combined therapeutic approach proposed is to deliver both IgY that neutralize critical aspects of the pathogenic potential of C. difficile and one or more probiotic microorganism that can help control the growth of C. difficile. The combination of these characteristics that can effectively prevent and/or treat C. difficile associated diarrhea.

The data indicate that we have been able to develop avian antibodies to a range of C. difficile antigens (listed in Table 1). Although all the antigens stimulated IgY antibodies as shown by reactivity in EIA and DFA, not all antibodies have toxin neutralization ability. However, there are two antigens that stimulated neutralization (shown by shaded area under Toxin A and Toxin B neutralization columns). Although there are Toxin B neutralizing antibodies that have been described, it is critical that a therapeutic treatment have the ability to neutralize both Toxin A and B to effectively interfere with the disease causing ability of the toxins. It is believed that there have been no published reports describing Avian IgY antibodies that can specifically neutralize Toxin A.

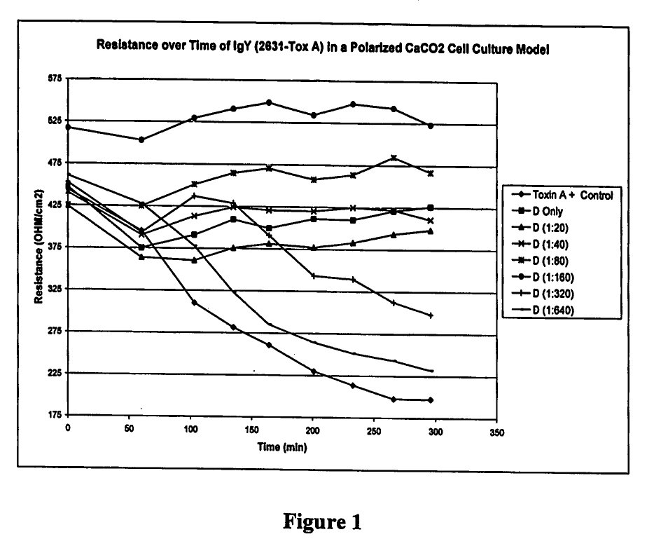

The dose response of neutralization of Toxin A is shown in FIG. 1. The Toxin A (+) control shows that in the CACO-2 cell monolayer there is a resistance drop over 300 minutes. When this same amount of Toxin A was mixed with Avian IgY that is reactive with Toxin A (2631), the ability of Toxin A to cause malfunctioning of the eukaryotic tight junctions is prevented. This “interference” prevents the toxin from damaging the tight junctions of the CACO-2 cells, so there is no drop in resistance (in the presence of Toxin A there is a drop in resistance). Testing up to 72 hours post-treatment confirmed that the Toxin A activity remained blocked even when tested at a 1:40 dilution of the IgY. This data indicates that the IgY neutralization of Toxin A was not a short-term transient effect.

The IgY preparations were unable to inhibit the growth of C. difficile. This may explain why avian IgY alone may not fully prevent or protect against C. difficile associated diarrhea, because without the added inhibitory effect of the probiotics, it may not be able to control the growth of C. difficile in the host. As such, even though there may be toxin neutralization, this does not address the underlying problem which is the overgrowth of C. difficile in the gut. The continued growth and output of toxin by C. difficile will eventually overwhelm the toxin neutralizing ability of the IgY.

Avian IgY antibodies that neutralize Toxin A and Toxin B are the ideal embodiment to use as reagents for the immunoglobulin (passive immunity) part of the combination therapeutic for C. difficile associated diarrhea.

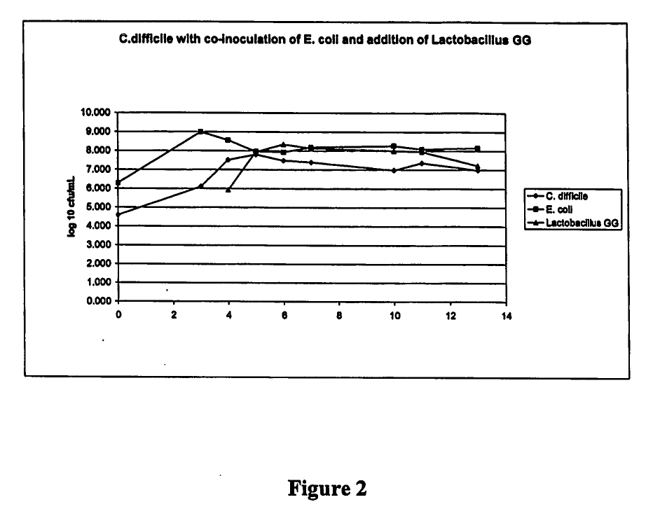

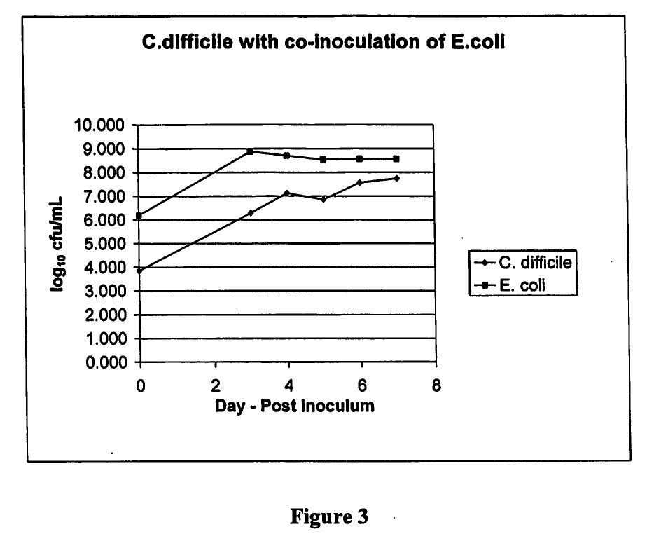

The second component of the combined therapeutic is the presence of probiotic microorganisms that will interfere with the growth of C. difficile. It has been reported that carriage of toxigenic C. difficile can occur without any disease as long as the host's normal flora keeps the level of toxigenic C. difficile under control. In a preferred embodiment, two to three microorganisms are used in combination as the probiotic arm of the therapeutic. As an example, FIG. 2 demonstrates the ability of Lactobacillus GG to reduce the level of C. difficile in our “Co-culture Reactor” model. In this model, a mixture of organisms is introduced and allowed to establish a stable level culture with C. difficile. Media is continuously fed into the reactor vessel and excess (waste) fluid is continuously allowed to drain. This Co-culture is a closed system that mimics what would happen in the host gut and provides a simulation of in vivo conditions. As shown in FIG. 3, C. difficile can maintain a stable level of organisms when grown in conjunction with Escherichia coli (common bowel organism). If Lactobacillus GG is introduced (FIG. 2), there is a resultant drop in the level of C. difficile by about one Log10. The probiotic organism alone cannot completely irradicate the C. difficile. This likely explains why in previous published data from clinical trials with Lactobacillus GG, there was variable ability of this probiotic organism to prevent and/or treat C. difficile associated diarrhea in humans. Even though the level of C. difficile may have been reduced, the Toxin A and Toxin B produced by the reduced level of organism would continue to damage the bowel mucosa (i.e. previous attempts to use probiotics only targeted one aspect of the C. difficile disease process).

In our combination therapeutic, control of C. difficile growth, neutralization of Toxin A and Toxin B as well as targeted delivery provides an optimal therapeutic despite the fact that either aspect alone cannot completely deal with the disease.

In the ideal combination therapeutic there will be ˜10⁸-10¹⁰ cfu of each of the selected probiotic microorganisms, as well as egg yolk product that contains IgY. The preferred embodiment will not use purified IgY, as the egg yolk containing the IgY will provide nutritional benefit for the probiotic organisms that are part of the combination therapeutic, as discussed above. This will enhance their ability to replicate and further control the levels of toxigenic C. difficile in the gut. As such, the combination therapeutic has a greater chance of being effective because it ensures all aspects of the disease causing ability of C. difficile have been targeted and provides a unique added advantage in that the egg yolk will provide nutrients for the probiotic organisms to ensure they are able to replicate. Although C. difficile can also utilize the nutritional components in egg yolk, it has a much slower growth rate compared to the probiotic organisms so they will have the advantage of utilizing the nutritional component faster than C. difficile thereby giving them an additional growth advantage.

As discussed above, delayed release encapsulation is the preferred embodiment for human treatment. This provides the advantage that the probiotics will not be damaged or killed by the gastric acidity when they pass through the stomach, thereby ensuring maximal dosing of the large bowel (where C. difficile disease occurs). In addition it will protect the nutritional value of the egg yolk product and ensure the IgY is not degraded by gut proteases prior to delivery to the optimal target site (large bowel). This approach has not previously been reported and the data provided indicate that neutralization of Toxin A and Toxin B can be achieved and that control of the growth of C. difficile can be achieved using probiotic organisms. The use of a combination of probiotics (multiple different organisms) will ensure that there is at least one of the organisms that can replicate in the host gut even if the host is receiving antibiotic therapy. In one embodiment, we have combined the use of hardy Gram positive bacteria (e.g. Lactobacillus species, or Bifidobacterium species), as well as a yeast (not inhibited by anti-bacterial agents) for the probiotic component of the combined therapeutic. These organisms can all survive drying and are very stable with respect to storage. In addition, antibiotics that affect bacteria will not kill yeast and anti-fungals that affect yeast will not kill bacteria. As such even in the presence of antibiotic or antifungal therapy, the probiotic therapeutic should have at least one organism that can still utilize the egg yolk nutrition and replicate rapidly to help inhibit the growth of C. difficile.

Clostridium difficile (C. difficile) is a spore-forming bacterial organism carried by many people that can become a cause of severe and even life-threatening diarrhea and colitis in patients whose normal intestinal ecosystems are disturbed by use of antimicrobial therapies.

C. difficile associated diarrhea (CDAD) usually occurs as a consequence of antibiotic therapy that is targeted at other diseases. The antibiotics disrupt normal intestinal flora, resulting in overgrowth of C. difficile organisms and the release of toxins that lead to diarrhea. Certain popular classes of antibiotics (ampicillin, cephalosporins, clindamycin and quinolones) particularly predispose patients to the development of C. difficile infection, but virtually every antimicrobial agent has been implicated.

The standard method of treatment for CDAD is the prescription of the antibiotics vancomycin or metronidazole. However the use of antibiotics as a therapy is problematic for the following reasons:

Cost—Standard treatment using vancomycin costs approximately $250.00 CDN per treatment. Metronidazole is considerably less expensive ($2.30 CDN per treatment), but may not be as effective.

Efficacy—Antibiotics act only on the C. difficile organism, not on its toxins or the spore form of the organism. This is because antibiotics can only act on a replicating organism. If the C. difficile organism sporulates, the spore survives in the gut in an inactive state and can germinate and recolonize after the antibiotic has left the system.

Cannot be used for prevention—Use of antibiotics to prevent disease has been shown to not be effective and has the added disadvantage of exposing gut organisms to the antibiotic, thereby increasing the chance that antibiotic resistance will be developed.

Disruption of intestinal flora—The antibiotics act on other intestinal organisms, prolonging the intestinal imbalance that led to the development of CDAD, and may therefore exacerbate the problem.

Microbial resistance—Overuse of antibiotics results in the emergence of microbial resistance, so physicians must ensure that treatment using antibiotics is reserved for severe or life-threatening cases only. Vancomycin is restricted in its use specifically for this reason. Even the alternative—metronidazole—has been implicated in the appearance of vancomycin resistant microorganisms

Increase in Immunocompromised Patients

A 1998 study identified the growth in the number of immunocompromised patients in US hospitals as a major factor contributing to the growing incidence of a variety of nosocomial infections in the US. This growth in the number of immunocompromised patients can be attributed to broad demographic trends (the aging of the general population), and to trends within the medical profession toward increasingly aggressive medical and therapeutic interventions, including implanted foreign bodies, organ transplantations, and xenotransplantations.

Additionally, the broad shift of surgical care from inpatient settings to outpatient centres has resulted in the sickest patients remaining in hospitals, increasing by default the concentration of immunocompromised patients within hospitals. Because the immunocompromised require therapeutic intervention—usually by means of antibiotics—to combat infection, this cohort is specifically predisposed to developing nosocomial C. difficile.

Growing Antibiotic Resistance

Antibiotic resistance is a growing problem that has recently been identified as a major public health threat and priority by several expert committees. Bacteria that resist not only single but multiple antibiotics have become increasingly widespread. In fact, according to the Centers for Disease Control and Prevention (CDC), virtually all significant bacterial infections in the world are becoming resistant to standard antibiotic treatments.

Several trends have been identified as contributing to the growing problem of antibiotic resistance:

Decline in development of new antibiotics—From 1945 until the late 1980s, new antimicrobial agents were developed faster than bacteria were able to develop resistance. Throughout the 1950's and 1960's, new classes of antibiotics were developed. However in the 1980's and early 1990's, research focused on improvement within existing classes of antibiotics, rather than on development of new classes. During this period, the development of new antibiotics slowed in favour of development of other drugs more economically attractive to pharmaceutical companies, thereby reducing the numbers of new antibiotics available in the market.

Overprescription of antibiotics by physicians—Antibiotics are routinely prescribed in hospitals to surgical patients and to immunocompromised patients (e.g. patients undergoing chemotherapy, HIV patients). Additionally, physicians frequently prescribe antibiotics to outpatients for conditions that may not warrant their use. Some sources estimate that tens of millions of prescriptions for antibiotics are written annually to treat viral illnesses for which these antibiotics offer no benefits. According to the Centers for Disease Control and Prevention (CDC), antibiotic prescribing in outpatient settings could be reduced by more than 30 percent without adversely affecting patient health. Reasons cited for overprescription of antibiotics by physicians include: time constraints do not permit appropriate diagnostic testing to identify better courses of action; and patients or consumers pressuring physicians, demanding antibiotics regardless of their appropriateness.

Increased Commercial Use of Antibiotics in Agriculture—Another much publicized concern is use of antibiotics in livestock, where the drugs are used in healthy food-producing animals to prevent disease and/or to increase weight gain, and the animals are later slaughtered for human consumption.

Increase in Resistance to Vancomycin

At the center of current concern regarding antimicrobial resistance is the antibiotic vancomycin, which for many infections is literally the drug of “last resort”. Some bacterial infections—for example, Methicillin-resistant Staphylococcus aureus (MRSA)—are treatable only with vancomycin. Highly virulent and increasingly antimicrobial resistant, MRSA has become a major source of nosocomial infections, and some strains of MRSA are resistant to all antibiotics except vancomycin. Recently, the first case of vancomycin resistance has been described for MRSA in Michigan, USA. Vancomycin resistance has now emerged in another common microorganism—Enterococcus—and there is great concern that unless vancomycin use is rigorously controlled, this resistance may be easily passed to Staphylococcus aureus. Specifically, metronidazole therapy is used first for CDAD to reduce the use of vancomycin in the gut, in an effort to reduce the risk of gut organisms such as Enterococcus developing vancomycin resistance.

CDAD is a ubiquitous and growing problem in hospitals in Canada, the US and Europe. Increasing numbers and concentrations of immunocompromised patients in Canadian and US hospitals suggest that growth in the incidence of CDAD will continue. This is particularly because the immunocompromised require antibiotics to defend against infection, and most antibiotics have been implicated in the onset of C. difficile associated diarrhea.

Benefits

Advantages of Avian Antibodies

The advantages of avian therapeutic antibodies over plasma-derived antibodies are:

Specificity—The specificity of the antibody content of plasma products is not controlled, and varies considerably among batches. In dried-egg products, however, the specificity of the antibodies is controlled, and it is possible to consistently produce antigen-specific antibodies.

Titer—The antibody titer of plasma products is also highly variable. In contrast, the antibody titer in egg yolk will be known, making it possible to use the dried-egg product for treatment of CDAD.

Advantages

Effectiveness—Therapeutic antibodies are a proven and effective means of neutralizing a number of pathogens, and pose a realistic alternative to antibiotics.

No possibility of microbial resistance—Microorganisms cannot develop resistance to antibodies.

Reduced microbial resistance pressure on vancomycin and metronidazole—By allowing the use of vancomycin and metronidazole to be reserved for more life-threatening infections, the invention will help to reduce the resistance pressure on those two drugs.

Targets all active components—Antibodies can be developed that can act on all four components of the disease: C. difficile organism, Toxins A and B, and the C. difficile spores.

No disruption to intestinal ecosystem—The therapy can be targeted at the pathogenic organisms without harming or disturbing other beneficial intestinal microorganisms.

Prophylaxis—The treatment may be used for both treatment and prevention of the disease.

Platform approach—By using a multi-faceted platform approach, the treatment has enhanced possibilities for neutralizing what is a complex, multi-faceted disease.

Typically, CDAD presents within 1 to 2 weeks after an antibiotic has been instituted, although presentation varies from 1 day to 6 weeks. The disease usually presents with profuse watery or mucoid diarrhea that may contain blood, abdominal pain, and low-grade fever, although symptoms range from only loose stools in the mildest cases to toxic megacolon or perforation in the most severe cases. Extraintestinal manifestations such as arthritis are rare. Dehydration, electrolyte depletion, and hypoproteinemia (from a protein-losing colonopathy) may occur with prolonged or severe disease.

Other complications include hemorrhage, sepsis, and pseudomembraneous colitis. Mortality is low (2%-5%), although it is higher in elderly or debilitated patients (10%-20%) or in those with fulminant colitis or toxic megacolon (30%-80%). In one study, the only factor associated with death was delay in the diagnosis of CDAD. In some patients (5%-19%), disease will be localized to the proximal colon. These patients may present with an acute abdomen, localized rebound tenderness, no diarrhea, and normal findings on sigmoidoscopy. Considering this diagnosis in such a patient with subsequent confirmation based on stool studies and computed tomography (CT) may help avoid unnecessary surgery. After recovery, patients may become asymptomatic carriers of C difficile, but most never have a relapse. However, 10% to 20% of patients will experience relapse regardless of the therapeutic agent used to treat CDAD. Such patients usually respond well to re-treatment with metronidazole or vancomycin but the risk of further recurrences may be as high as 65%.

In general, C difficile is noninvasive, although rare cases of intestinal tissue invasion have been reported in children with malignancy or a compromised immune system. The development of CDAD requires an alteration in normal gut flora or mucosal immunity, acquisition and germination of spores, overgrowth of C difficile, and toxin production. The most important toxins are toxin A (enterotoxin) and toxin B (cytotoxin). Toxin A binds to mucosal receptors and causes cytotoxicity by disrupting cytoplasmic microfilaments. Toxin B then enters the damaged mucosa and causes further toxicity, resulting in hemorrhage, inflammation, and necrosis.

The toxins interfere with protein synthesis, attract granulocytes, and increase capillary permeability and peristalsis. In patients with severe disease, inflammation may involve deep layers, resulting in toxic dilatation or perforation.

While the preferred embodiments of the invention have been described above, it will be recognized and understood that various modifications may be made therein, and the appended claims are intended to cover all such modifications which may fall within the spirit and scope of the invention.

TABLE 1
Antibodies (IgY) produced in Avian Host:

| | Reactivity by | | | Inhibition |
|---|---|---|---|---|
| | EIA or DFA to | Toxin A | Toxin B | of |
| | antigen used for | neutral- | neutral- | C. difficile |
| Antigen: | immunization | ization | ization | replication |
| C. difficile | Positive | Negative | Negative | Negative |
| 2631 | | | | |
| whole cells | | | | |
| C. difficile | Positive | Negative | Negative | Negative |
| 29975 whole | | | | |
| cells | | | | |
| C. difficile | Positive | Negative | Negative | Negative |
| CM26 | | | | |
| whole cells | | | | |
| C. difficile | Positive | Negative | Negative | Negative |
| 2631 spores | | | | |
| C. difficile | Positive | Negative | Negative | Negative |
| 29975 Spores | | | | |
| C. difficile | Positive | Negative | Negative | Negative |
| CM26 | | | | |
| Spores | | | | |
| C. difficile | Positive | Positive | Negative | Negative |
| 2631 | | | | |
| Toxin A | | | | |
| C. difficile | Positive | Negative | Negative | Negative |
| 2631 | | | | |
| Toxin B | | | | |
| C. difficile | Positive | Negative | Negative | Negative |
| 29975 Toxin A | | | | |
| C. difficile | Positive | Negative | Positive | Negative |
| 29975 Toxin B | | | | |
| C. difficile | Positive | Negative | Negative | Negative |
| CM26 Toxin A | | | | |
| C. difficile | Positive | Negative | Negative | Negative |
| CM26 Toxin B | | | | |

1. A pharmaceutical composition comprising:
polyclonal antibodies directed against at least one enteric pathogen; and
a probiotic.

2. The pharmaceutical composition according to claim 1 wherein the enteric pathogen is selected from the group consisting of: Aeromonas hydrophilia, Bacillus cereus, Vibrio parahemolyticus, Vibrio cholerae O1, Vibrio cholerae non-O1, Vibrio vulnificus, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinia enterocolitica, Yersinia pestis, Yersinia pseudotuberculosis, Plesiomonas shigelloides, Listeria monocytogenes, enteric viruses, rotavirus, Norwalk-like viruses, enteric adenoviruses, coronavirus and all other non-enveloped enteroviruses, and enteric parasites and fungi, Cryptosporidium, and Cyclospora.

3. The pharmaceutical composition according to claim 1 wherein the probiotic is selected from the group consisting of: Lactobacilli species, Bifidobacteria species, Saccharomyces species, Enterococci species, Eubacteria species and mixtures thereof.

4. The pharmaceutical composition according to claim 1 wherein the polyclonal antibodies are egg yolk antibodies.

5. The pharmaceutical composition according to claim 1 including an oligosaccharide.

6. The pharmaceutical composition according to claim 1 wherein the pharmaceutical composition is microencapsulated.

7. The pharmaceutical composition according to claim 1 wherein the polyclonal antibodies are raised against more than one antigen derived from the enteric pathogen.

8. A method of preparing a pharmaceutical composition comprising:
admixing a polyclonal antibodies directed against at least one enteric pathogen; and a probiotic.

9. The method according to claim 8 wherein the enteric pathogen is selected from the group consisting of: Aeromonas hydrophilla, Bacillus cereus, Vibrio parahemolyticus, Vibrio cholerae O1, Vibrio cholerae non-O1, Vibrio vulnificus, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinia enterocolitica, Yersinia pestis, Yersinia pseudotuberculosis, Plesiomonas shigelloides, Listeria monocytogenes, enteric viruses, rotavirus, Norwalk-like viruses, enteric adenoviruses, coronavirus and all other non-enveloped enteroviruses, and enteric parasites and fungi, Cryptosporidium, and Cyclospora.

10. The method according to claim 8 wherein the probiotic is selected from the group consisting of: Lactobacilli species, Bifidobacteria species, Saccharomyces species, Enterococci species, Eubacteria species and mixtures thereof.

11. The method according to claim 8 wherein the polyclonal antibodies are egg yolk antibodies.

12. The method according to claim 8 including adding an oligosaccharide.

13. The method according to claim 8 including microencapsulating the pharmaceutical composition.

14. The method according to claim 8 wherein the polyclonal antibodies are raised against more than one antigen derived from the enteric pathogen.

15. A method of treating or preventing a gastrointestinal illness comprising administering to a patient in need thereof an effective dose of a pharmaceutical composition comprising:
polyclonal antibodies directed against at least one enteric pathogen; and
a probiotic.

16. The method according to claim 15 wherein the enteric pathogen is selected from the group consisting of: Aeromonas hydrophilia, Bacillus cereus, Vibrio parahemolyticus, Vibrio cholerae O1, Vibrio cholerae non-O1, Vibrio vulnificus, Salmonella enterica, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (ETEC, EPEC, EHEC, EaggEC, UPEC and EIEC), Campylobacter jejuni, Campylobacter coli, Campylobacter lari, Campylobacter fetus, Yersinia enterocolitica, Yersinia pestis, Yersinia pseudotuberculosis, Plesiomonas shigelloides, Listeria monocytogenes, enteric viruses, rotavirus, Norwalk-like viruses, enteric adenoviruses, coronavirus and all other non-enveloped enteroviruses, and enteric parasites and fungi, Cryptosporidium, and Cyclospora.

17. The method according to claim 15 wherein the probiotic is selected from the group consisting of: Lactobacilli species, Bifidobacteria species, Saccharomyces species, Enterococci species, Eubacteria species and mixtures thereof.

18. The method according to claim 15 wherein the polyclonal antibodies are egg yolk antibodies.

19. The method according to claim 15 wherein the pharmaceutical composition includes an oligosaccharide.

20. The method according to claim 15 wherein the pharmaceutical composition is microencapsulated.

21. The method according to claim 15 wherein the polyclonal antibodies are raised against more than one antigen derived from the enteric pathogen.
